SEQUENCE LISTING (8 sequences)

SEQ ID NO: 1 - DNA, 35 bp, unknown
  Mca1617F primer
aaggagatataccaatgacaaaagaagaatttgaa

SEQ ID NO: 2 - DNA, 28 bp, unknown
  Mca1617R primer
tattcattacgctcctcttggctgagcg

SEQ ID NO: 3 - DNA, 42 bp, unknown
  T7 universal primer
gaaattaatacgactcactatagggagaccacaacggtttcc

SEQ ID NO: 4 - DNA, 43 bp, unknown
  T7 universal primer
ctctagaaataattttgtttaactttaagaaggagatatacca

SEQ ID NO: 5 - DNA, 34 bp, unknown
  Mca1616F primer
gttctgcagttaaggagtagagccatggctattg

SEQ ID NO: 6 - DNA, 36 bp, unknown
  Mca1616R pimer
gttgaattcagatctgtcgcgtgtcgagcgcccgaa

SEQ ID NO: 7 - DNA, 44 bp, unknown
  Mca1617F primer
gttgctagcgtaaggaggtacatatgacaaaagaagaatttgaa

SEQ ID NO: 8 - DNA, 27 bp, unknown
  Mca1617R primer
gttggatccgacaactagctccggctt

(19) United States
(12) Patent Application Publication
(10) Pub. No.: US 2006/0014179 A1
(43) Pub. Date: Jan. 19, 2006

(54) Inferring function from shotgun sequencing data

(75) Inventor: Richard J. Roberts, Wenham, MA (US)

Correspondence Address: New England Biolabs, Inc.;Attn: Harriet M. Strimpel, D.Phil, 32 Tozer Road, Beverly, MA, 01915, US

(73) Assignee: New England Biolabs, Inc., Beverly, MA (US)

(21) Appl. No.: 11/142,790

(22) Filed: Jun. 1, 2005

Related U.S. Application Data
(60) Provisional application No. 60/576,196, filed on Jun. 2, 2004.

(51) Int. Cl. C12Q 1/68; G06F 19/00

(52) U.S. Cl. 435/6; 702/20

(57) ABSTRACT

Methods are described for detecting genes that encode toxic proteins using maps derived from shotgun libraries by determining the presence of gaps in clone start sites on either side of open reading frames. The method is exemplified by identifying a previously unknown restriction endonuclease gene.

### CROSS REFERENCE

This application gains priority from U.S. provisional application Ser. No. 60/576,196 filed Jun. 2, 2004, herein incorporated by reference.

### BACKGROUND

Toxic proteins can be found in all genomes and serve a variety of functions. Many microbial genomes express toxic proteins known as restriction endonucleases that vary widely between different isolates and have significant utility in biomedical research. A single bacterial genome may contain several restriction endonucleases some of which are active and some of which are not. One clue to finding genes that encode restriction endonucleases, which share little or no sequence homology with one another, is their spatial juxtaposition to genes encoding methyltransferases. The latter genes can be identified using bioinformatics approaches because of the existence of conserved sequence motifs. (U.S. Pat. No. 6,383,770 and 6,689,573).

Even if open reading frames (ORFs) are identified in the vicinity of genes encoding methyltransferases, there are no sequence identifiers for ORFs encoding a restriction endonuclease. Moreover, without cloning, it has not been possible to determine if a putative restriction endonuclease is active or a mutant.

Indeed mutations leading to inactive genes are quite common (Kong et al. Nucl. Acids Res. 28: 3216-3223 (2000); Lin et al. Proc. Natl. Acad. Sci. USA 98: 2740-2745 (2001)). It would be highly desirable to have a bioinformatics method that could reliably identify restriction enzyme genes that are capable of giving active restriction enzymes. This would then permit cloning and biochemical analysis to be done in the most effective fashion.

Shotgun libraries have been widely used for genome sequencing. The genomic DNA is broken into fragments of approximately 2000 bases by mechanical shearing, restriction endonuclease cleavage, non-specific nucleases or by chemical methods. The fragments are then cloned into vectors and a host cell, most commonly E. coli, is then transformed with these vectors. The vectors are then replicated and clones are formed. A library typically contains about 25,000 clones (see Table 1). A single strand of the duplex genomic DNA in these clones may then be sequenced to provide reads which are then assembled into a contig map. These genome maps can be found in public databases. The shotgun libraries from which the map is derived are commonly stored.

### SUMMARY

In one embodiment of the invention, a method is provided for identifying whether an ORF encodes a toxic protein. The method includes the steps of: a) obtaining an in silico map of clones from a shotgun library aligned on a target DNA sequence; (b) detecting a gap in the map corresponding to a numerical deficiency or lack of start sites of shotgun clones in a region such that there is a statistically underrepresented number or lack of clones spanning the ORF; and (c) determining whether a protein product of the ORF is a toxic protein.

In an embodiment of the invention, the region starts within one end of the ORF and extends away from the ORF. For example, a clone start site may lie within a few nucleotides from the end of an ORF such that the clone extends over the ORF but does not express an active protein. This clone start site may then represent the boundary of the gap in start sites extending over the ORF, which represents sequences encoding a functional toxic protein that cannot be cloned.

In certain embodiments, the target DNA fragment is a genome, more particularly a genome obtained from a bacterium, an archaea or a virus. In additional embodiments, the toxic protein is a restriction endonuclease encoded by an ORF adjacent to a methylase.

In an additional embodiment, a method includes an additional step of expressing the ORF in vivo or by in vitro transcription/translation.

### DESCRIPTION OF THE FIGURES

FIG. 1 (a) shows a schematic representation of a section of a genome containing a hypothetical restriction endonuclease (R) and a methyltransferase (M) gene. The overlapping clones allow the determination of the sequence of the genome section. The sequence for the complete R gene is predicted to be absent within any single clone because of the toxic nature of the expression product.

FIG. 1(b) shows a cartoon of the location of gaps around an ORF indicating a toxic gene where the shotgun clones are assumed to average 2000 base pairs in length. (7) corresponds to a 1000 bp toxic gene. (8) corresponds to 850 base pairs in the putative toxic gene required for expression of the toxic protein. (9) corresponds to a gap in clone starts on the top strand of the duplex genomic DNA. (10) corresponds to a gap in clone starts on the bottom strand of the duplex genomic DNA. (11) corresponds to the 5′ and 3′ boundaries of the top strand gap (10) while (12) corresponds to the 5′ and 3′ boundaries on the bottom strand gap (9). The size of the gene and the portion required for expression of a toxic protein are hypothetical examples and are not intended to represent a limitation on size. The actual values will vary according to different genes.

FIG. 2 shows a flow diagram of the computational analysis of the shotgun sequence reads.

FIG. 3(a) shows the distribution of clone starts from clones in a shotgun library across a region of the Hemophilus influenzae genome known to encode the restriction endonuclease HindIII. (1) and (2) mark the location of the gap. As predicted, the gaps at locations on opposing sides of the ORF on the top and bottom strands reflect the presence of a restriction endonuclease gene (HindIII) that is toxic to the E. coli host. Each bar represents the start site of a shotgun clone on one strand of the target DNA which extends in a direction 5′ to 3′.

FIG. 3(b) shows a schematic representation of a distribution of shotgun clone reads across the region of the Hemophilus influenzae genome shown in FIG. 3(a). The dark lines correspond to aligned sequences and the light grey lines correspond to non-aligned sequences. Vt denotes a gap in the distribution of clone starts mapped to the top strand of the DNA and Vb denotes a gap in the distribution of clone starts mapped to the bottom strand of the DNA.

FIG. 4 shows the distribution of clone starts from clones in a shotgun library across a region of the Methanococcus jannaschii genome known to encode MjaII. (3) and (4) mark the location of the gap. As predicted, the gaps at locations on opposing sides of the ORF on top and bottom strand reflect the presence of a restriction endonuclease gene (MjaII) that is toxic to the E. coli host. The two clone start sites mapped within the gap correspond to mutant clones that cannot express protein.

FIG. 5 shows the distribution of clone starts from clones in a shotgun library across a region of the Methylococcus capsulatus genome believed to encode a methyltransferase (M.McaTORF1616P) with an ORF followed by a vsr DNA mismatch endonuclease. (5) and (6) mark the location of the gap. Cloning of the ORF region between the gap and the putative methyltransferase and testing the clones for gene activity showed that the ORF encodes a restriction enzyme. In vitro transcription/translation of these sequences additionally confirmed that the ORF between M.McaTORF1616P and vsr mismatch endonuclease is an active restriction endonuclease.

FIG. 6 shows an agarose gel image of the endonuclease activity of Mcal617. Lanes are annotated as: M, 2-log DNA ladder; 1, λDNA only; 2, λDNA+2 μl IVT mixture without DNA template; 3, λDNA+2 μl IVT reaction mixture with Mcal617 PCR product; 4, λDNA+2 μl IVT reaction mixture with Mcal617 PCR product, supplemented with 1× NEB buffer 2; 5, λDNA+2 μl IVT mixture with Mcal617 PCR product, supplemented with 1× NEB buffer 4 (New England Biolabs, Inc., Beverly, Mass.).

FIG. 7 shows Mcal617 endonuclease activity in a crude tract. The lanes are as follows:

Lanes 1 and 7: lambda-HindIII and PhiX-HaelII size standards (New England Biolabs, Inc., Beverly, Mass.).

Lane 2: 9 μl crude extract/50 μl reaction;

Lane 3: 3 μl crude extract/50 μl reaction;

Lane 4: 1 μl crude extract/50 μl reaction;

Lane 5: 0.3 μl crude extract/50 μl reaction;

Lane 6: 0.1 μl crude extract/50 μl reaction.

FIG. 8 shows Mcal617 Endonuclease cleavage activity compared with BssHII cleavage activity.

Lanes 1 and 5: lambda-HindIII and PhiX-HaeIII size standards (New England Biolabs, Inc., Beverly, Mass.);

Lane 2: λDNA cut with Mcal617;

Lane 3: λDNA cut with Mcal617 and BssHII;

Lane 4: λDNA cut with BssHII.

### DETAILED DESCRIPTION OF EMBODIMENTS

A bioinformatic method is provided that is capable of identifying active restriction enzyme genes and thus directing the most efficient molecular characterization of such genes. This provides a means to discover restriction endonucleases with new specificities.

The following terms are defined for use in the specification and in the claims where applicable.

The term “toxic protein” refers to a protein which when expressed in a host cell causes the host cell to become nonviable or causes cell death.

The term “host cell” refers to any cell that can be transformed by foreign DNA where the foreign DNA may be a plasmid or vector containing a gene and the gene can be expressed in the cell.

The term “shotgun library” refers to a set of clones containing DNA fragments randomly generated by fragmentation of a genome or large DNA and cloned in a suitable host organism usually E. coli. Shotgun sequencing involves sequencing the DNA fragments inserted in the clones. The genome or large DNA may be from a eukaryote including a human, mammal or plant, or from a prokaryote, virus or archaea. There is no limitation as to the source of the genome or DNA fragment. Nor is there an upper limitation on size of DNA along which shotgun libraries are mapped. It is understood that if each shotgun DNA fragment is 2000 bases, the size of the DNA or genome to which the shotgun fragments are to be mapped will be larger than 2000 bases. The method described herein takes advantage of a large amount of potentially useful information that is discarded after shotgun libraries have been prepared and utilized for genome sequencing. As stated above, the significance of clones in a shotgun library for the present analysis relates to mapping the start sites of the clones.

The shotgun library will contain fragments that represent the entire sequence about 5-20 times (see Table 1 for example). Because the initial preparation of fragments is usually done in a random fashion, the random sequence data that is produced needs to be reassembled in much the same way that a jigsaw is put back together. It has been confirmed that the clone starts and hence the sequences derived from the clones are substantially random and evenly distributed around the genome. It is here shown that the random pattern can be disrupted when an ORF encoding a toxic protein is present in the genome.

The term “gap” refers to a region of the target DNA fragment where there is an absence of clone start sites. In those circumstances where no single clone spans an ORF and a gap in clone starts is found, there is a presumption that the ORF encodes a protein that is toxic to the host cell. An ORF surrounded by two such gaps on the appropriate strands would then be surmised to encode a protein toxic to the host in which it was cloned. The gap may however be interrupted by a statistically underrepresented number of clones or by even a single clone. These one or more clone start sites may correspond to clones, which are presumed to contain mutations that destroy the function of the expressed protein. Examples of such mutations include frame shifts, truncations, deletions, translation-blocking mutants or chimeras including fusions to foreign sequences.

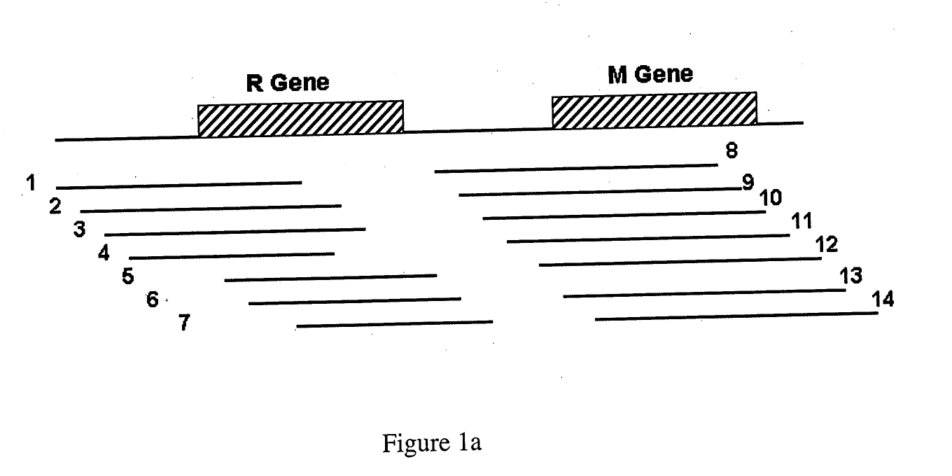

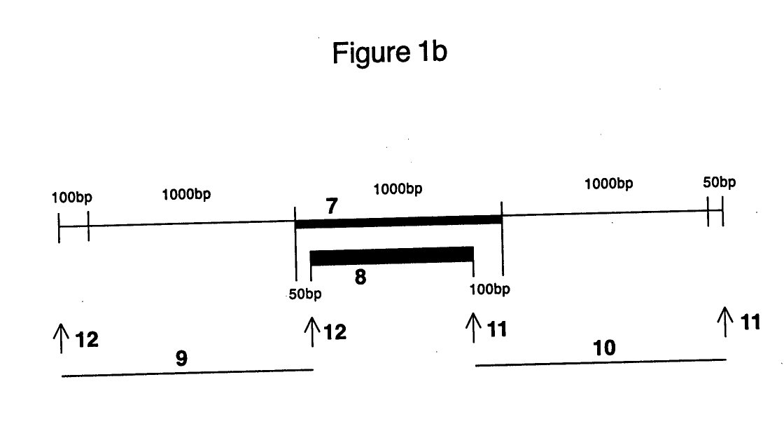

A gap may be identified by two boundary clone start sites where one boundary of the gap is represented by a clone start site lying a few nucleotides within an ORF and extending so that it contains most, but not all, of the ORF and the second boundary is represented by a clone start site lying many nucleotides away from the ORF, but which defines a clone that is not long enough to contain the entire ORF (FIG. 1b).

The term “read” refers to a sequence corresponding to approximately 500 base pairs in an approximately 2000 bp fragment from a shotgun library. Not all of the sequence for a 2000 bp fragment can be reliably determined in a single sequencing event. The approximately 500 bp fragment in a read is the sequence from a single sequencing event that can be most reliably determined. A significant feature of a read is that it establishes the start site of the clone. Knowing the existence of a clone and mapping its start site is more significant than the exact length or the sequence of the read. In some instances the actual sequence is relevant when it shows the presence of mutations that destroy function or chimeric clones containing foreign DNA that also destroy function.

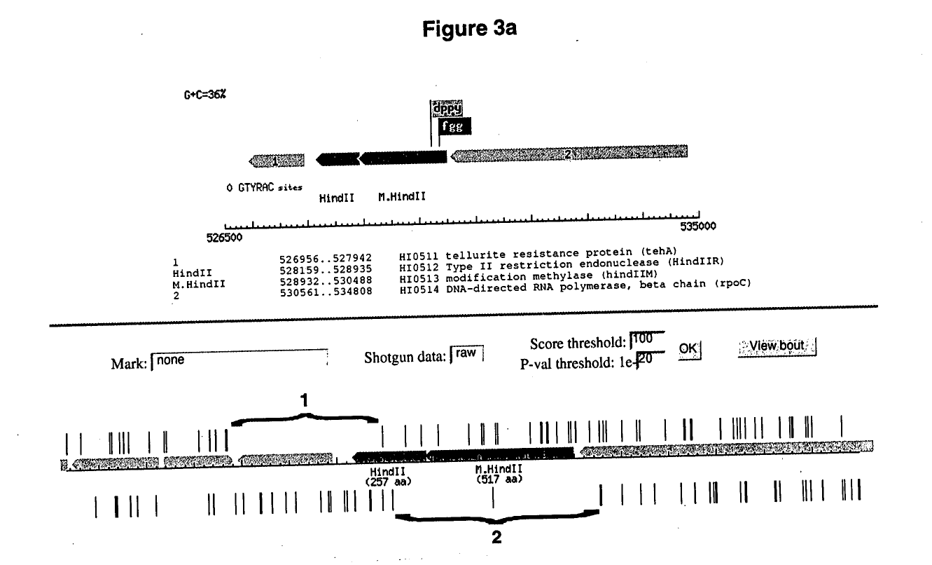

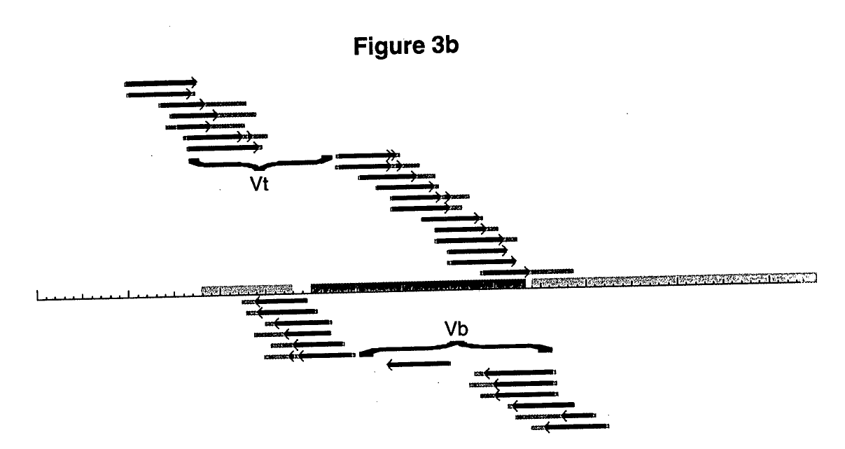

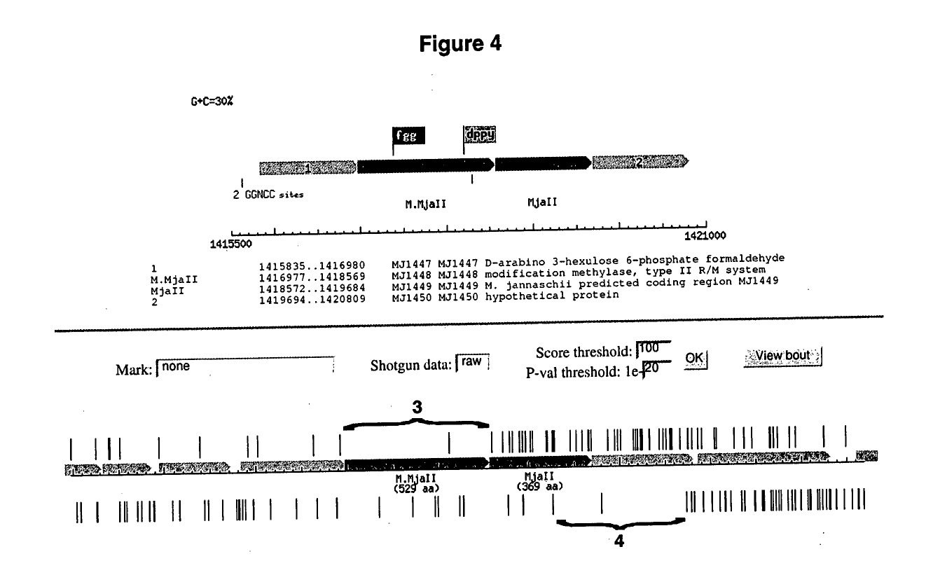

The above observations have been tested and confirmed for test DNA genomes known to contain restriction endonucleases. However, it is expected that the general approach is also applicable to other toxic proteins. In FIGS. 3-4, a characteristic gap is observed for the ORFs expressing Hemophilus influenza HindIII and Methanococcus jannaschii MjaII on the top strand and the bottom strand where the gap extends into the ORF. The single clones, marked in the clone map corresponding to the bottom strand in both HindIII and MjaII genes, contain mutations that would render the expressed proteins non-functional.

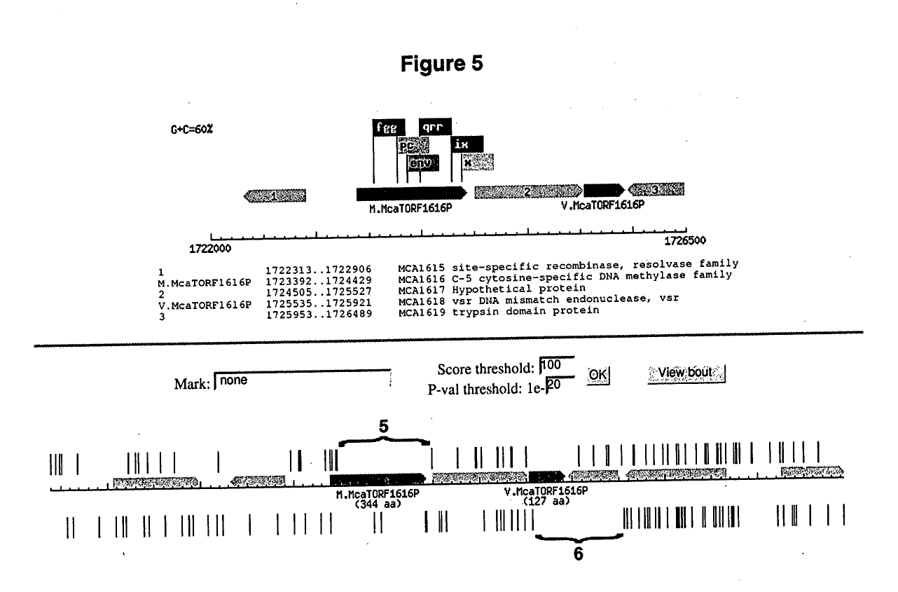

The methodology has further been tested for the genomic DNA of Methanococcus capsulata not previously analyzed for toxic genes (FIG. 5). In FIG. 5, the gaps were identified as indicated and subsequently shown to encode a restriction endonuclease by in vitro transcription/translation (Example 1) and cloning (Example 2).

The present functional methods using shotgun libraries to identify ORFs encoding toxic proteins are robust. The Figures and Examples demonstrate the utility of this approach for discovering novel restriction endonuclease proteins. An advantage of this approach is the direct measurement of functionality. Traditionally, ORFs thought to encode toxic proteins such as restriction endonucleases were identified by their sequence characteristics such as sequence homology to a known toxic protein or location adjacent to another gene such as a methyltransferase. Formerly these sequences would then be cloned and expressed to determine functionality under conditions that could be quite problematic owing to the toxic nature of the gene products. Not all ORFs adjacent to a methylase were found to encode active restriction endonucleases. For example, the ORF encoding a putative restriction endonuclease adjacent to the M.HindV ORF (HI1041 in the H. influenzae genome) has been found to be inactive. This could be readily predicted by shotgun cloning maps using the present methods.

Data Analysis

The original reads from a shotgun sequence experiment typically contain stretches of 400-500 nucleotides of DNA sequence which represent the ends of longer pieces of cloned DNA, usually 1,500 to 2,000 nucleotides. A bacterial shotgun library generally contains at least 25,000 clones. Examples are provided in Table 1 for three bacterial strains.

The analysis of reads to identify potentially lethal genes is carried out as follows:

The end of each sequence read is mapped to its appropriate location within the finished complete genome sequence using a search algorithm such as BLASTN (Altschul, S. F., et al. J. Mol. Biol. 215: 403 (1990)). Each ORF from the completed genome sequence is checked against the full collection of sequence reads and the ends of the sequence reads are mapped on to the ORF and its flanking sequences. This is repeated for all of the ORFs in the genome sequence. In this way, the start sits and approximate spans of the shotgun sequences can be determined and will result in a mapping of the shotgun library onto the original sequence as exemplified in FIGS. 1 through 5.

The locations of all identified ORFs are checked against the mapped sequence reads. Sequence reads are often inaccurate, but an occasional sequence error is unimportant. What is significant is that the read confirms that a clone exists.

Occasionally, one can expect that a clone start provides a clone spanning a presumed lethal gene because the cloned sequence contains an inactivating mutation. Although this is rare, it may occur from time to time. Consequently, the intact ORF is a candidate for a lethal gene. For instance, in the case of the R and M genes shown in the schematic in FIG. 1a, none of the clones contain the R gene completely within them, whereas the M gene is represented (FIG. 1a, reads 9 to 14). Thus the R gene is a candidate for a lethal gene.

It should be noted that this procedure is most effective for ORFs that are shorter than the average size of the clones from which the sequence reads are obtained. Where the ORFs are longer than about 2000 bp, data from a second collection of shotgun reads with a longer average insert size can be used. Such sets of longer reads may be available because libraries with larger inserts, such as 8-10 kb, are made to help close gaps in the original sequence.

This process is repeated for all ORFs in a genome fragment or whole genome to provide a list of candidate lethal genes. Of special interest for the discovery of restriction endonucleases are those ORFs that either lie immediately adjacent to a methyltransferase gene or no more than one ORF away. These are the preferred candidates for restriction enzyme genes.

If one of the fragments from the shotgun sequencing contains a complete toxic enzyme gene, it will not be clonable because the expression product would be lethal to the host cell. Hence, examination of the raw data from the original shotgun reads that are used to clone and assemble the genome sequence display discontinuities corresponding to ORFs in the genome. These ORFs correspond to toxic genes such as deoxyribonucleases, ribonucleases, certain proteases and other kinds of hydrolytic enzymes that are not usually found in E. Coli or other host cells and yet have a substrate present in the host cytoplasm.

For example, a bacterial genome cloned in a host cell such as E. coli with a map assembled accordingly may produce clones with intact M genes but the clones corresponding to the flanking regions where restriction enzymes would be expected do not contain a complete ORF for the lethal restriction enzyme. Accordingly, the functional map of the genome will contain a gap corresponding to a lack of a clone start in this region of the genome. Occasionally, a clone expressing a restriction endonuclease may be obtained if the restriction endonuclease gene contains a mutation that renders the restriction endonuclease inactive. In these circumstances, there would be no gap and the complete gene would be clonable. An advantage of the method described above is that the non-clonable sequence is immediately functionally identified assuming that all non-toxic genes are represented in a shotgun library.

A toxic gene, here exemplified by a restriction endonuclease, can be identified by the following method:

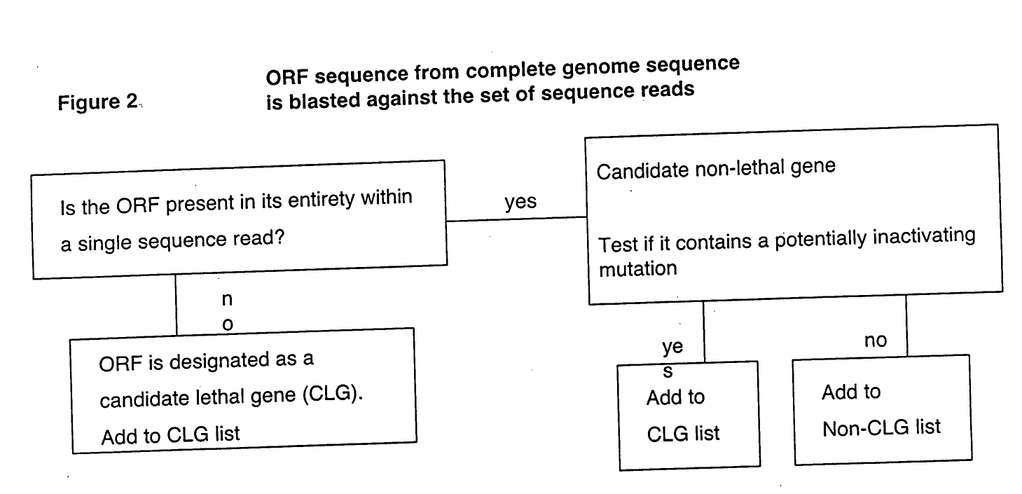

(I) The data from a shotgun sequencing experiment is analyzed (FIG. 2). From this data, it is possible to predict which ORFs, flanking a given DNA methytransferase gene, are the best candidates to encode a restriction enzyme gene.

(II) Once a candidate restriction endonuclease gene is identified from analysis of the shotgun data, the gene is tested experimentally by a two-step cloning procedure in which first the methyltransferase gene is cloned in a vector resulting in complete methylation of the host, and second the restriction endonuclease gene is cloned into that same host (see Example 2). Additionally, a procedure for cloning using, for example, pLTK7, is described in U.S. Pat. No. 6,689,573 herein incorporated by reference.

The methodology described herein involving the analysis of shotgun sequencing data provides strong predictive power when used in combination with genetic information present in the art and optionally bioinformatics techniques for identifying the sequence and location characteristics of toxic genes including candidate restriction-modification systems.

All references cited herein are incorporated by reference, including U.S. provisional application Ser. No. 60/576,196.

TABLE 1

| | H. influenzae | H. pylori | M. jannashii |
|---|---|---|---|
| Number of | 26,883 | 25,769 | 39,521 |
| clones | | | |
| Read Length | 462 | 547 | 479 |
| av. bp | | | |
| Total sequence | 12.5 | 14 | 19 |
| (Mb) | | | |
| Genome size | 1.83 | 1.66 | 1.66 |
| (Mb) | | | |
| Coverage | 6.7 | 8.4 | 11 |
| Gap length | 68 | 64 | 42 |
| average | | | |

### EXAMPLES

### Example 1

### Demonstration that the ORF Identified with Gaps in Shotgun Sequence Clone Starts for M. capsulatus is a Functional Restriction Endonuclease

1. In Vitro Transcription/Translation of Mcal617

The ORF of Mcal617 was first amplified from genomic DNA of Methylococcus capsulatus using primers Mcal617F and Mcal617R (Table 2). Using the first PCR product as template, the second PCR was performed to append the T7 promoter and ribosomal binding site at its 5′ end using primers T7_universal and Mcal617R (Table 2). The PCR product was purified using QIAGEN Quick PCR Purification kit and its concentration was determined to be 40 ng/μl. Both PCR were performed using the high-fidelity Phusion polymerase (Finnzymes.com, Espoo, Finland). All primers were synthesized at New England Biolabs, Inc., Beverly, Mass.).

The coupled in vitro transcription/translation (IVT hereafter) was performed using PURESYSTEM (Post Genome Institute Co., Ltd., Tokyo, Japan). A 10 μl reaction was assembled using 7 μl IVT mixture, 11 μl PCR product and 2 μl water. The reaction mixture was incubated at 37° C. for 2 hours to allow in vitro translation.

2. Endonuclease Activity Assay

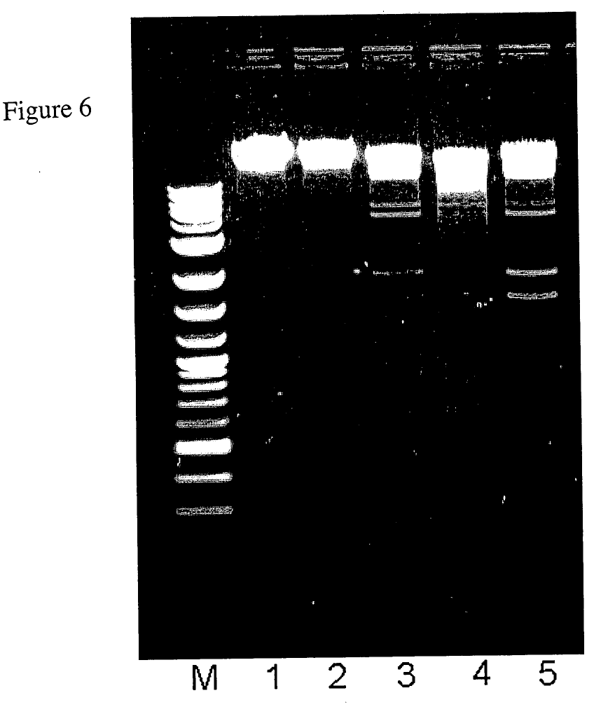

The endonuclease activity of in vitro translated Mcal617 was tested upon the digestion of phage λDNA (New England Biolabs, Inc., Beverly, Mass.). 1 μg phage λDNA (at concentration of 0.2 μg/μl) was digested with 2 μl IVT mixture and was incubated at 37° C. for 1.5 hours. 1 μl RNase A (Qiagen, Valencia, Calif.) at concentration of 0.1 μg/1 μl was then added and the reaction mixture was further incubated at 37° C. for 30 minutes. The digestion reaction mixture was then analyzed by electrophoresis in a 1% agarose gel (FIG. 6).

3. Results

As shown in FIG. 6, the IVT mixture with Mcal617 PCR product exhibits endonuclease activity by cutting EDNA to distinct bands (lane 3,4,5, FIG. 6), while the IVT mixture itself does show such ability (lane 2, FIG. 6). The residual EDNA is due to incomplete digestion from the limited translated product of Mcal617.

TABLE 2

Primers used in PCR

| primer | |
|---|---|
| name | Primer sequence |
| Mca1617F | AAGGAGATATACCAATGACAAAAGAAGAATTTGAA |
| | (SEQ ID NO:1) |
| Mca1617R | TATTCATTACGCTCCTCTTGGCTGAGCG |
| | (SEQ ID NO:2) |
| T7 | GAAATTAATACGACTCACTATAGGGAGACCACAACGGT |
| universal | TTCC (SEQ ID NO:3) |
| | CTCTAGAAATAATTTTGTTTAACTTTAAGAAGGAGATA |
| | TACCA (SEQ ID NO:4) |

### Example 2

### Expressing the M. capsulatus Endonuclease encoded by the Mcal617 ORF

Primers were designed to amplify the putative methyltransferase, ORF Mcal616, and the putative endonuclease, Mcal617. The forward primers incorporate a restriction site to facilitate cloning, a ribosome binding site, an NdeI restriction endonuclease site at the ATG start of translation codon for Mcal617, and sequence matching the M. capsulatus genomic DNA. The reverse primers have restriction sites to facilitate cloning. The primers synthesized were:

(SEQ ID NO: 5)

Mca1616 Forward
5′-GTTCTGCAGTTAAGGAGTAGAGCCATGGCTATTG-3′

(SEQ ID NO:6)

Mca1616 Reverse
5′-GTTGAATTCAGATCTGTCGCGTGTCGAGCGCCCGAA-3′

(SEQ ID NO:7)

Mca1617 Forward
5′-GTTGCTAGCGTAAGGAGGTACATATGACAAAAGAAGAATTTGAA-3′

(SEQ ID NO:8)

Mca1617 Reverse
5′-GTTGGATCCGACAACTAGCTCCGGCTT-3′

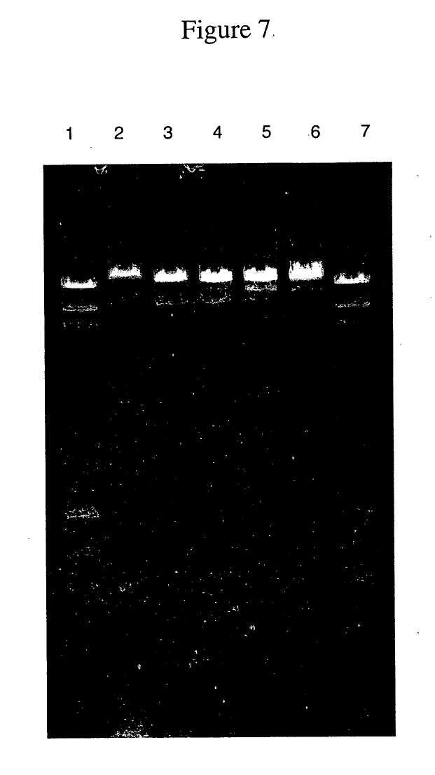

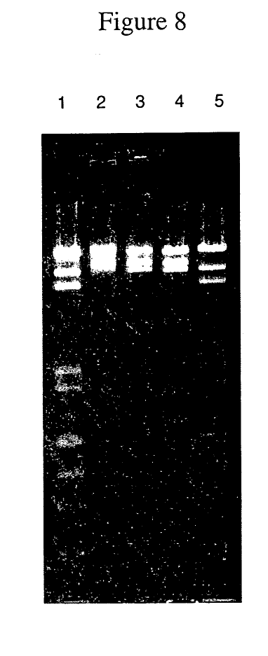

Genomic DNA was isolated from M. capsulatus cells using a bead beating kit (MoBio, Inc, Solana Beach, Calif.). As a first attempt at expressing this R-M system, both genes were amplified together using primers Mcal616 forward (SEQ ID NO:5) and Mcal617 reverse (SEQ ID NO:8) using Taq DNA polymerase under standard conditions with a hot start. The amplified product was purified over a “DNA Clean and Concentrate” spin column following the manufacturer's instructions (ZYMO Research, Orange, Calif.). The purified DNA was digested with PstI and BamHI under standard conditions and again purified using the spin columns. This DNA was then ligated to pUC19 vector previously cut with PstI and BamHI and dephosphorylated. The ligated vector was then transformed into ER2683 chemically competent cells and the transformed cells were grown overnight on LB+ampicillin plates. Approximately 650 colonies were obtained. The colonies were scraped off the plate and placed in 1.5 ml sonication buffer (20 mM Tris, 1 mM DTT, 0.1 mM EDTA pH7.5) and disrupted by sonication. The extract was centrifuged at 16,000 g for 10 minutes and the supernatant was assayed for restriction endonuclease by serial dilution of the extract in NEBuffer2 containing λDNA at 20 μg/ml (FIG. 7). Fragmentation of the λDNA was observed, indicating the presence of a restriction endonuclease activity. The crude extract was applied to a 1 ml HiTrap Q HP column (Amersham Biosciences, Upsala, Sweden). The column was eluted with a step gradient of NaCl in Sonication Buffer and endonuclease activity was observed in the 250 mM NaCl and 300 mM NaCl steps. The partially purified endonuclease was used to map cut sites in pUC-AdenoBC4 and pUC-AdenoXba DNAs (these DNAs are pieces of Adeno2 DNA inserted into pUC19). The positions of cleavage were consistent with the endonuclease cutting at GCGCGC sites, which is the recognition sequence of BssHII. Lambda DNA was digested with the Mcal617 endonuclease, with BssHII, and with the two enzymes together. If the Mcal617 enzyme cuts at BssHII sites, the pattern for the two enzymes together should be the same as that of either enzyme alone. The pattern for BssHII alone and for BssHII and Mcal617 together is the same (FIG. 8). There was not enough Mcal617 enzyme to give a complete digest, so the pattern for Mcal617 alone represents a partial digest pattern. Interestingly, the single GCGCGC site in PhiX174 DNA is not detectably cut by the Mcal617 enzyme preparation, although it is cut by BssHII. This indicates a difference between Mcal617 and BssHII.

Stable Expression of Mcal617 Endonuclease

To stably express the Mcal617 endonuclease, the methylase is first introduced into cells to allow the cell's DNA to be protectively modified, after which the endonuclease gene is introduced under controlled regulation on a second, compatible vector.

To express this restriction modification system in E. coli, the Mcal616 methyltransferase ORF was amplified with primers 1 and 2 using Taq polymerase under standard conditions with a hot start. The Mcal617 putative endonuclease ORF was amplified with primers 3 and 4 as above. The amplified products were purified over a “DNA Clean and Concentrate” spin column following the manufacturer's instructions (ZYMO Research, Orange, Calif.). The purified DNA for the methyltransferase (Mcal616) was then digested with PstI and BglII under standard condition and again purified using the spin columns. This DNA was then ligated to pUC19 vector previously cut with PstI and BamHI and dephosphorylated. The ligated vector and Mcal616 ORF DNA was transformed into ER2566 chemically competent cells and the transformed cells were grown on LB+ampicillin plates. Ten individual transformants were grown and a miniprep of their plasmid DNA was prepared. The plasmid DNA of each was cut with PvuII to see if the Mcal616 ORF was present. 8 of 10 transformants examined had the Mcal616 ORF inserted into the pUC19 vector.

These Mcal616 containing cells are then grown and made chemically competent by standard methods. The amplified DNA of the putative endonuclease gene (ORF Mcal617) is cut with NdeI and BamHI and spin column purified. The DNA is then ligated into a controlled expression vector, such as pSAPV6, previously cut with NdeI and BamHI, dephosphorylated and purified. This vector, pSAPV6 (U.S. Pat. No. 5,663,067) has the T7 controlled expression system, enhanced by the addition of multiple transcription terminators upstream and downstream of the T7 promoter. The ligated putative endonuclease and vector is then transformed into the ER2566 cells carrying the putative methyltransferase ORF. Individual transformants are then examined for the presence of the Mcal617 endonuclease DNA in the pSAPV6 vector, and those having the DNA are grown to late log phase and induced with 0.3 mM IPTG for 2 hours. The cells are then harvested and a lysate prepared by sonication. Such cell extracts are examined for endonuclease activity by mixing various amounts of the lysate with lambda DNA in NEBuffer 4 and incubating at 37* for one hour, then examining the reactions for DNA fragments on agarose gels.

What is claimed is:

1. A method for identifying an open reading frame (ORF) encoding a toxic protein, comprising:
a) obtaining an in silico map of a plurality of shotgun clones from a shotgun library aligned on a target DNA sequence;
(b) detecting a gap in the map corresponding to a numerical deficiency in start sites of the shotgun clones in a region such that there is a statistically underrepresented number of clones spanning the ORF; and
(c) determining whether a protein product of the ORF is a toxic protein.

2. A method according to claim 1, wherein the region starts at approximately one end of the ORF and extends away from the ORF.

3. A method according to claim 1, wherein the target DNA fragment is a genome

4. A method according to claim 3, wherein the genome is a selected from a bacterial genome, an archaeal genome and a viral genome.

5. A method according to claim 3, wherein the toxic protein is a restriction endonuclease.

6. A method according to claim 3, wherein the toxic gene is mapped to an ORF adjacent to a methylase.

7. A method according to claim 6, wherein the step of identifying the gene expressing the toxic protein from the ORF further comprises expressing the ORF in vivo or by in vitro translation.

8. A method for identifying an open reading frame (ORF) encoding a toxic protein, comprising:
a) obtaining an in silico map of shotgun clones from a shotgun library aligned on a target DNA sequence;
(b) detecting a gap in the map corresponding to a lack of start sites of the shotgun clones in a region such that there is a lack of clones spanning the ORF; and
(c) determining whether a protein product of the ORF is a toxic protein.
